(19) United States
(12) Patent Application Publication
(10) Pub. No.: US 2024/0347168 A1
(43) Pub. Date: Oct. 17, 2024

(54) SYSTEMS AND METHODS FOR GENERATING A PERSONALIZED TARGET COMBINATION OF ITEMS FOR A USER

(71) Applicant: Huawei Cloud Computing Technologies Co., Ltd., Guian (CN)

(72) Inventors: Shiqi HE, Xiangjiang Century City (CN); Rindranirina RAMAMONJISON, Coquitlam (CA); Yong ZHANG, Richmond (CA)

(21) Appl. No.: 18/134,905

(22) Filed: Apr. 14, 2023

(51) Int. Cl. G16H 20/60; G06N 3/02

(52) U.S. Cl. CPC G16H 20/60; G06N 3/02

(57) ABSTRACT

Methods and systems for generating a personalized target combination of items for a user are disclosed. The method includes accessing an item database, accessing a target score database, receiving information about item election values of the user, determining, for each item of the item database, an item election value based on the first input, receiving information about a user profile of the user, determining a set of user target feature scores for the user, determining a list of candidate item combinations from the item database, determining, for each candidate item combination, a global feature score based on the feature scores of the sub-items of the items of the candidate item combination, receiving an indication of a preferred candidate item combination and adjusting a respective quantity of the sub-items of the items of the preferred item combination to obtain the personalized target item combination by minimizing an objective function.

### FIELD OF THE TECHNOLOGY

The present technology relates to computer-implemented recommendation systems. In particular, servers and methods for generating a personalized target combination of items are disclosed for the illustrative application of personalized optimization-based meal plan recommendation.

### BACKGROUND

According to World Health Organization (WHO), non-communicable diseases, such as cardiovascular diseases, diabetes and cancer, kill 41 million people each year, equivalent to 74% of all deaths globally. These diseases are preventable through effective interventions that tackle shared risk factors, such as unhealthy diets. Several technologies have been developed to help the users cultivating a healthy diet habit. For example, recipe recommendation systems which aimed at designing meal plans for users with a specific dish or meal in mind, or knowledge-based recommendation framework using expert-validated meals for healthy diets have been proposed.

However, it seems that these technologies fail to provide a comprehensive solution for meal recommendation. Some solutions do not consider user preferences and lack personalization features, which requires the evaluation of the system directly from real users in real-time.

There is thus a desire for a system that can provide a personalized target combination of items, or “meals”, for a flexible daily meal plan based on nutritional needs.

### SUMMARY

Implementations of the present technology have been developed based on developers' appreciation of shortcomings associated with the prior art.

In a first broad aspect of the present technology, there is provided a computer-implemented method for generating a personalized target combination of items for a user. The computer-implemented method includes accessing an item database comprising information about a plurality of items, each item being associated with one or more sub-items composing the item, each sub-item being associated with one or more feature scores for one or more corresponding features of the sub-item, accessing a target score database comprising information about target feature scores for a plurality of pre-determined user categories, receiving a first input, from a user device associated with the user, comprising information about item election values of the user, the item election values being indicative of a preference of the user for a first set of items, determining, for each item of the item database, an item election value based on the first input, receiving a second input, from the user device, comprising information about a user profile of the user, determining, based on a matching score between the user profile and a given one of the pre-determined user categories of the target score database, a set of user target feature scores for the user, determining a list of candidate item combinations from the item database, each candidate item combination comprising a plurality of items, determining, for each candidate item combination, a global feature score based on the feature scores of the sub-items of the items of the candidate item combination, receiving, from the user device, an indication of a preferred candidate item combination among the candidate item combinations and updating a respective quantity of the sub-items of the items of the preferred item combination to obtain the personalized target item combination by minimizing:

$${\sum }_{A ∈ Features} \frac{\vert {R}^{A} - {\sum }_{i = 1}^{n} {X}_{i} \times {Y}_{i}^{A} \vert}{{R}^{A}}$$

where Xᵢ is a quantity of the sub-item i, Yᵢ^{A} is a feature value of the sub-item i for the feature A and R^{A} is a target feature score for the feature A as defined in the target score database for the user.

In some non-limiting embodiments, the computer-implemented method further includes ordering the list of candidate item combinations based on global election value of the candidate item combinations, the global election value of each candidate item combination being determined based on election values associated with items of the candidate item combination and providing to the user device a top portion of the list of candidate item combinations.

In some non-limiting embodiments, the computer-implemented method further includes updating the item election values of the items of the item database based on the indication of the preferred item combination among the candidate item combinations.

In some non-limiting embodiments, determining, for each item of the item database, an item election value based on the first input includes employing a neural network to determine, based on the item election values of the first set of items, an item election value for each of the other item of the item database.

In some non-limiting embodiments, the neural network is employed to determine a sub-item election value for each sub-item of the item database.

In some non-limiting embodiments, determining the list of candidate item combinations from the item database includes selecting a second set of items, the items of the second set having a corresponding election score above a pre-determined threshold.

In some non-limiting embodiments, each candidate item combination comprises three items.

In some non-limiting embodiments, each item of the item database comprises a tag indicative of an item category of the item, the item database comprising three categories of items.

In some non-limiting embodiments, each item maps information about a meal, each sub-item maps information about an ingredient of the meal, each feature score maps information about a nutrition value of the corresponding ingredient, and each feature maps information about a nutrient of the corresponding ingredient.

In some non-limiting embodiments, the computer-implemented method further includes transmitting information about the personalized target item combination to the user.

In a second broad aspect of the present technology, there is provided a system for generating a personalized target combination of items for a user, the system includes a controller and a memory storing a plurality of executable instructions which, when executed by the controller, cause the controller to access an item database comprising information about a plurality of items, each item being associated with one or more sub-items composing the item, each sub-item being associated with one or more feature scores for one or more corresponding features of the sub-item, access a target score database comprising information about target feature scores for a plurality of pre-determined user profiles, receive, from a user device associated with the user, a first input comprising information about item election values of the user, the item election values being indicative of a preference of the user for a first set of items, determine, for each item of the item database, an item election value based on the first input, receive, from the user device, a second input comprising information about a user profile of the user, determine, based on a matching score between the user profile and a given one of the pre-determined user profiles of the target score database, a set of user target feature scores for the user, determine a list of candidate item combinations from the item database, each candidate item combination comprising a plurality of items, determine, for each candidate item combination, a global feature score based on the feature scores of the sub-items of the item of the candidate item combination, receive, from the user device, an indication of a preferred candidate item combination among the candidate item combinations and update a respective quantity of the sub-items of the items of the preferred item combination to obtain the personalized target item combination by minimizing:

$${\sum }_{A ∈ Nutrients} \frac{\vert {R}^{A} - {\sum }_{i = 1}^{n} {X}_{i} \times {Y}_{i}^{A} \vert}{{R}^{A}}$$

where Xᵢ is a quantity of the sub-item i, Yᵢ^{A} is a feature value of the sub-item i for the feature A and R^{A} is a recommended feature score for the feature A as defined in the target score database for the user.

In some non-limiting embodiments, the system further orders the list of candidate item combinations based on global election value of the candidate item combinations, the global election value of each candidate item combination being determined based on election values associated with items of the candidate item combination and provides to the user device a top portion of the list of candidate item combinations.

In some non-limiting embodiments, the system further adjusts the item election values of the items of the item database based on the indication of the preferred item combination among the candidate item combinations.

In some non-limiting embodiments, determining, for each item of the item database, an item election value based on the first input includes employing, by the controller, a neural network to determine, based on the item election values of the first set of items, an item election value for each of the other item of the item database.

In some non-limiting embodiments, the neural network is employed to determine a sub-item election value for each sub-item of the item database.

In some non-limiting embodiments, determining the list of candidate item combinations from the item database includes selecting, by the controller, a second set of items, the items of the second set having a corresponding election score above a pre-determined threshold.

In some non-limiting embodiments, each item combination comprises three items.

In some non-limiting embodiments, each item of the item database includes a tag indicative of an item category of the item, the item database comprising three categories of item.

In some non-limiting embodiments, each item maps information about a meal, each sub-item maps information about an ingredient of the meal, each feature score maps information about a nutrition value of the corresponding ingredient, and each feature maps information about a nutrient of the corresponding ingredient.

In some non-limiting embodiments, the system further transmits information about the personalized target item combination to the user.

In the context of the present specification, a “server” is a computer program that is running on appropriate hardware and is capable of receiving requests (e.g., from client devices) over a network, and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expression “at least one server”.

In the context of the present specification, “user device” is any computer hardware that is capable of running software appropriate to the relevant task at hand. Thus, some (non-limiting) examples of user devices include personal computers (desktops, laptops, netbooks, etc.), smartphones, and tablets, as well as network equipment such as routers, switches, and gateways. It should be noted that a device acting as a user device in the present context is not precluded from acting as a server to other user devices. The use of the expression “a user device” does not preclude multiple user devices being used in receiving/sending, carrying out or causing to be carried out any task or request, or the consequences of any task or request, or steps of any method described herein. It is contemplated that the user device and the server can be implemented as a same single entity.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, the expression “component” is meant to include software (appropriate to a particular hardware context), firmware, hardware, or a combination thereof, that is both necessary and sufficient to achieve the specific function(s) being referenced.

In the context of the present specification, the expression “computer usable information storage medium” or “computer-readable medium” is intended to include media of any nature and kind whatsoever, including RAM, ROM, disks (CD-ROMs, DVDs, floppy disks, hard drivers, etc.), USB keys, solid state-drives, tape drives, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a schematic representation of an item combination recommendation environment in accordance with an embodiment of the present technology;

FIG. 2 is a schematic representation of an item database in accordance with an embodiment of the present technology;

FIG. 3 is a schematic representation of a target feature score database in accordance with an embodiment of the present technology;

FIG. 4 is a diagram showing transmission of information between a recommendation system and a user device within the item combination recommendation environment of FIG. 1 in accordance with an embodiment of the present technology;

FIG. 5 is a block diagram of modules of the server of FIG. 1 for determining a personalized target item combination for a user of the user device in accordance with an embodiment of the present technology;

FIG. 6 is a diagram showing operations of a target score module of the recommendation system of FIG. 1;

FIG. 7 is a table showing items associated with election values for the user in accordance with an embodiment of the present technology;

FIG. 8 is a diagram showing operations of a neural network to determine election values according to some implementations of the present technology;

FIG. 9 is a diagram showing operations of an item combination generating module of the recommendation system of FIG. 1;

FIG. 10 is a table showing a list of candidate item combinations in accordance with an embodiment of the present technology;

FIG. 11 is a representation of an interface for presenting a portion of the list of candidate item combinations in accordance with an embodiment of the present technology;

FIGS. 12A and 12B are flow diagrams showing operations of a method for generating a personalized target combination of items for a user in accordance with an embodiment of the present technology; and

FIG. 13 is a block diagram of an electronic device in accordance with an embodiment of the present technology.

It should also be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagram herein represents conceptual views of illustrative circuitry embodying the principles of the present technology. Similarly, it will be appreciated that any flowcharts, flow diagrams, state transition diagrams, pseudo-code, and the like represent various processes that may be substantially represented in non-transitory computer-readable media and so executed by a computer or processor, whether or not such computer or processor is explicitly shown.

The functions of the various elements shown in the figures, including any functional block labeled as a “processor” or “processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general-purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

In the context of the present disclosure, the term “nutrient” refers to a substance used by a living entity (e.g. human entity) to live, such as protein and vitamin. The term “nutrition requirement”, or “target feature score” used for a meal plan recommendation application, both recommended value and tolerable upper intake level of a nutrient. The terms “nutrition fact”, or “feature score” used for a meal plan recommendation application, both refers to a nutrition values in an ingredient or a meal. The terms “nutrition database”, or “target score database” used for a meal plan recommendation application, both refers to a database that contains nutrition requirements. The terms “meal”, or “item” used for a meal plan recommendation application, both refers to data including information about a corresponding meal, a meal name, recipe and ingredients. The terms “meal type”, or “item category” used for a meal plan recommendation application, both refers to category of the meal, such as for example and without limitation, breakfast, lunch and dinner. The terms “meal database”, or “item database” used for a meal plan recommendation application, both refers to a database that contains meal information.

Broadly speaking, the illustrative example of an application of the present technology is the personalized optimization-based meal plan recommendation application. However, other applications are contemplated in alternative implementations for recommendation of any type of items such as clothing, real estate, movies or sport training exercises.

With these fundamentals in place, we will now consider some non-limiting examples to illustrate various implementations of aspects of the present technology.

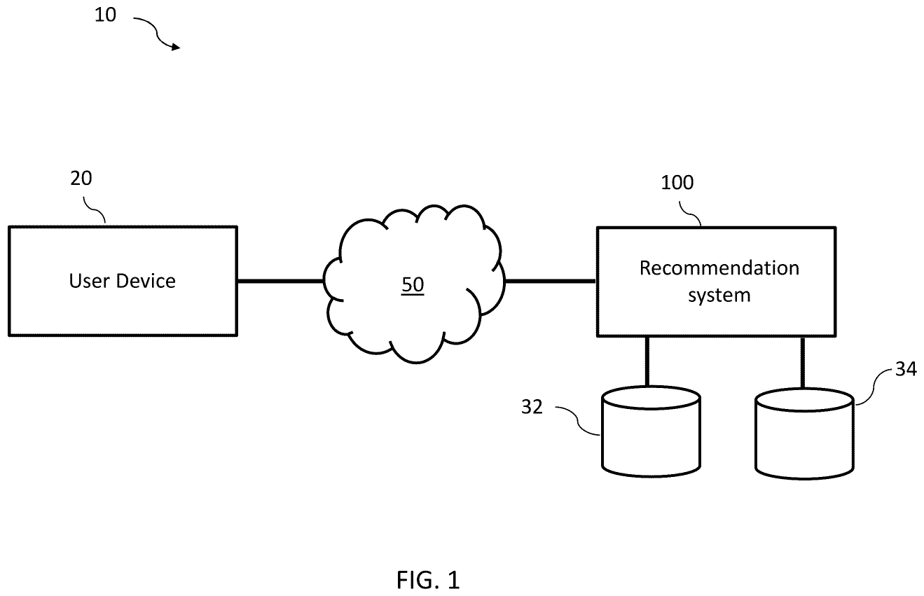

Referring to FIG. 1, there is shown a schematic diagram of an item combination recommendation environment 10, the item combination recommendation environment 10 including a recommendation platform operated by a recommendation system 100. The recommendation system may be a server 100. The recommendation system 100 is thus suitable for implementing non-limiting embodiments of the present technology. It is to be expressly understood that the item combination recommendation environment 10 as depicted is merely an illustrative implementation of the present technology. Thus, the description thereof that follows is intended to be only a description of illustrative examples of the present technology. This description is not intended to define the scope or set forth the bounds of the present technology. In some cases, what is believed to be helpful examples of modifications to the item combination recommendation environment 10 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the Item combination recommendation environment 10 may provide in certain instances simple implementations of the present technology, and that where such is the case they have been presented in this manner as an aid to understanding. As persons skilled in the art would understand, various implementations of the present technology may be of a greater complexity.

Generally speaking, the recommendation system 100, by operating the recommendation platform, enables a user associated with a user device to be provided with a personalized and optimized meal plan recommendation that may be an item combination (e.g. a combination of three meals that fulfill personalized nutrition requirements and user preferences). Broadly speaking, the recommendation system 100 may determine, based on a user profile and pre-determined target feature scores, a combination of item based on characteristics of said item and sub-items composing the item.

More specifically, with reference to FIG. 1, the item combination recommendation environment 10 includes the recommendation system 100 that host, operate and run the recommendation platform, and a user device 20 associated with a user desiring to be provided with a personalized and optimized personalized target item combination. The user device 20 is communicatively coupled to the recommendation system 100 over a communication network 50 via any wired or wireless communication link including, for example, 4G, LTE, Wi-Fi, or any other suitable connection. In some non-limiting implementations of the present technology, the communication network 50 may be implemented as the Internet. In other implementations of the present technology, the communication network 50 can be implemented differently, such as any wide-area communication network, local-area communication network, a private communication network and the like. How the communication links between the user device 20 and the recommendation system 100 are implemented will depend inter alia on how the user device 20 and the recommendation system 100 are implemented.

The item combination recommendation environment 10 also includes a first database 32, or “item database 32”. In some implementations, the item database 32 includes information about a plurality of items, each item being associated with one or more sub-items composing the item, each sub-item being associated with one or more feature scores for one or more corresponding features of the sub-item.

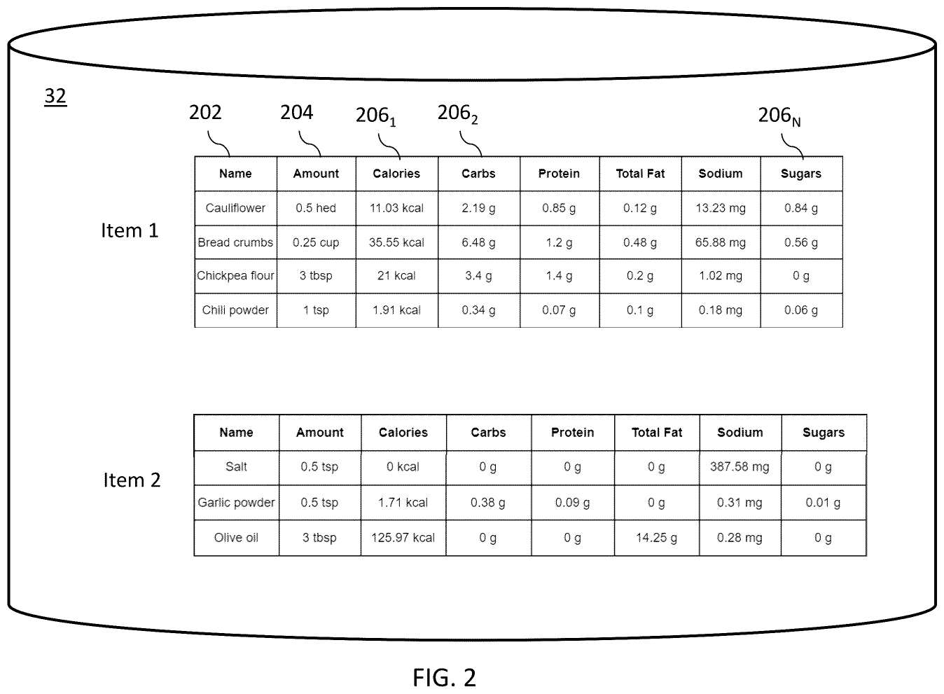

More specifically and as best shown on FIG. 2, the item database 32 includes information about a plurality of items, two of which are being depicted on FIG. 2. Each item is associated with one or more sub-items 202 that collaboratively form the item 202. Each sub-item 202 is associated with a quantity to form the corresponding item, and one or more feature score 206ᵢ (206₁ to 206_{N} on FIG. 2) for a corresponding one or more feature of the sub-item 202. In this example, each item of the item database 32 maps information of a meal, and each sub-item 202 of the item is an ingredient. The quantity 204 of a sub-item is a default amount of the ingredient in the meal and the features of each ingredient are nutrients. The nutrients may be macronutrients such as carbohydrate, protein, fat, linoleic acid, fiber, water, elements such as iron, calcium, chromium, sodium, zinc, iodine and/or vitamins such as vitamin A, vitamin B6, vitamin B12, vitamin C and vitamin D. The feature scores are thus, in this illustrative example, indicative of a quantity of the nutrients for the corresponding quantity 204 to form the corresponding item.

It can be said that the item database 32 includes information about a plurality of sub-item with corresponding feature scores for feature for a default quantity of the sub-item, the sub-item being grouped to form a plurality of items. Two different items may include a same sub-item.

In some implementations, each item of the item database 32 includes a tag indicative of an item category of the item. For example and without limitation, three categories of items may be defined in the item database 32 (e.g. breakfast, lunch and dinner).

As will be described in greater details hereinafter, each item of the item database is associated with an item election value for a corresponding user, an item election values being indicative of a user preference of the user for a corresponding item. For example and without limitation, the item election values may be between 0 and 1, 0 being indicative of a dislike of the item by the user, and 1 being indicative of a liking of the item by the user. For example, a given item A may have an item election value of 0.7 for a first user and an item election values of 0.2 for a second user.

The item combination recommendation environment 10 also includes a second database 34, or “target feature score database 34”. In some implementations, the target feature score database 34 includes information about target feature scores for a plurality of pre-determined user profiles.

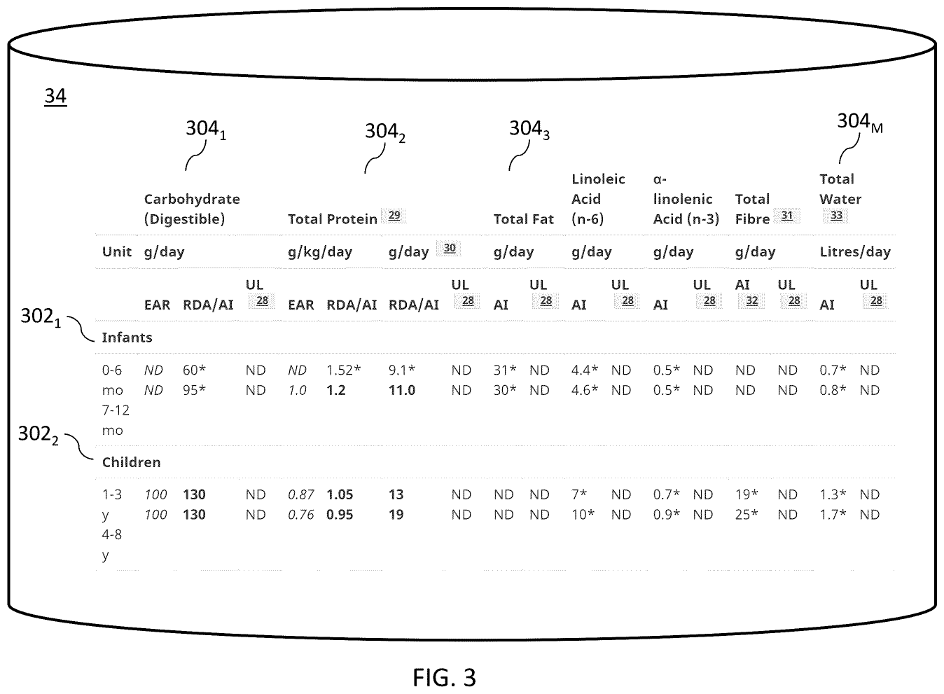

More specifically and as best shown on FIG. 3, the target feature score database 34 includes information about a plurality of pre-determined user categories such as user category 302₁ “infant” and user category 302₂ “Children”. Other user categories are contemplated but no shown for clarity of FIG. 3, such as “Female Children 8 years old”, “Male Teenager Children 8 years old”, “Female Children 9 years old”, “Male Teenager Children 9 years old”, “Pregnancy 18-30 Years Old”, etc. Each user category is associated with target feature scores for a plurality of corresponding features 304₁ to 304_{M}. In some implementations, the features 304₁ to 304_{M} correspond to the features 206₁ to 206_{N} or be derived therefrom.

In some implementations, an operator of the server or a service provider providing the recommendation service prepare and populate the target feature score database 34 prior usage of the recommendation system 100. For example, the target feature score database 34 may be imported from government nutrition guidelines. These guidelines usually give the recommended nutrition values for different groups of people. In generally, a comprehensive guideline should consider these factors for different people: 1) age; 2) gender; 3) height; 4) weight; 5) physical activity level.

In the depicted non-limiting implementation of the present technology, the databases 32, 34 are depicted as two separate and independent databases. In alternative non-limiting implementations of the present technology, information of the databases 32, 34 are included in a single data base such that the databases 32, 34 are a same database. The database 32 in FIG. 2 may be populated and/or updated by a third-party data provider and/or imported by an operator of the recommendation system 100. The database 34 in FIG. 3 may be populated and/or updated based on third-party data such as, in this illustrative example, nutrition guidelines provided by national and/or international health organizations. Databases 32, 34 may be updated by re-importing data from third-party data providers regularly (e.g. once a month).

In the depicted non-limiting implementation of the present technology, the recommendation system 100 is a single server. In alternative non-limiting implementations of the present technology, the functionality of the recommendation system 100 may be distributed and may be implemented via multiple servers or be performed by another data processing device communicably connected thereto.

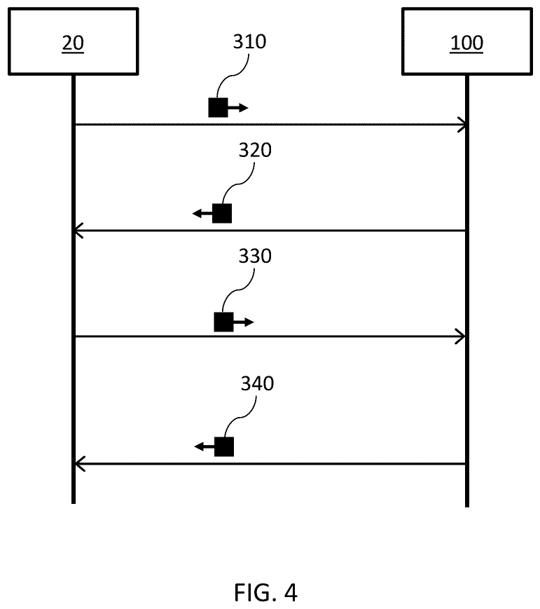

Broadly speaking and with reference to FIG. 4, communication between the user device 20 and the recommendation system 100 for determining a personalized target item combination for the user includes transmitting information 310 about a user profile of the user from the user device 20 to the recommendation system 100. The user profile may include information about age, gender, weight, socio-economic status or any other suitable information about the user. Based on the user profile, the recommendation system 100 determines a user category for the user and fetches target feature scores for the user category in the target score database.

In some implementations, the determination of the personalized target item combination includes an initialization step for determining election values of the items of the item database for the user. More specifically, the recommendation system 100 receives a first input from the user device 20 including information about item election values of the user indicative of a preference of the user for a first set of items. For example and without limitation, a survey may be presented to user in the form of pairs of item images (e.g. two meal images). The user is asked to select the preferred item between these two images. The survey may repeat ten times (20 item images in total) and the user will make 10 choices in total. In some implantations, there are three item categories (e.g. breakfast, lunch and dinner). Therefore, by considering all three meal types, the use may be presented with 30 surveys and make 30 choices.

Each time the user makes a selection, the recommendation system 100 updates item election values of the presented items in the item database 32 accordingly. The recommendation system 100 may employ a neural network to compute the user preference (i.e. the item election values) for each item of the item database 32 based on the selections made by the user on the first set of items. Determination and update of an item election value distribution will be described in greater details hereinafter.

In some other implementation, the initialization step may be performed by omitting the surveys and setting the item election values of the items of the item database 32 to a pre-determined value (e.g. 0.5).

The recommendation system 100 further determines a list of candidate item combinations based on the target feature scores and election values of the items for the user. Determination of the list of candidate item combinations is described in greater details hereinafter. Information 320 about the list of candidate item combinations is further transmitted by the recommendation system 100 to the user device 20.

The user may further transmit information 330 about an indication of a preferred item combination among the candidate item combinations from the user device 20 to the recommendation system 100. For example, the user may select the candidate item combination C1 using the interface 1000. In response, the recommendation system 100 updates a respective quantity of the sub-items of the items of the preferred item combination to obtain the personalized target item combination. In some implementations, the recommendation system 100 does so by minimizing the following objective function:

$${\sum }_{A ∈ Features} \frac{\vert {R}^{A} - {\sum }_{i = 1}^{n} {X}_{i} \times {Y}_{i}^{A} \vert}{{R}^{A}} \qquad ( 1 )$$

where Xᵢ is a quantity of the sub-item i, Yᵢ^{A} is a feature value of the sub-item i for the feature A and R^{A} is a target feature score for the feature A as defined in the target score database for the user.

The recommendation system 100 may further transmit information 340 about the personalized target item combination to the user device 20.

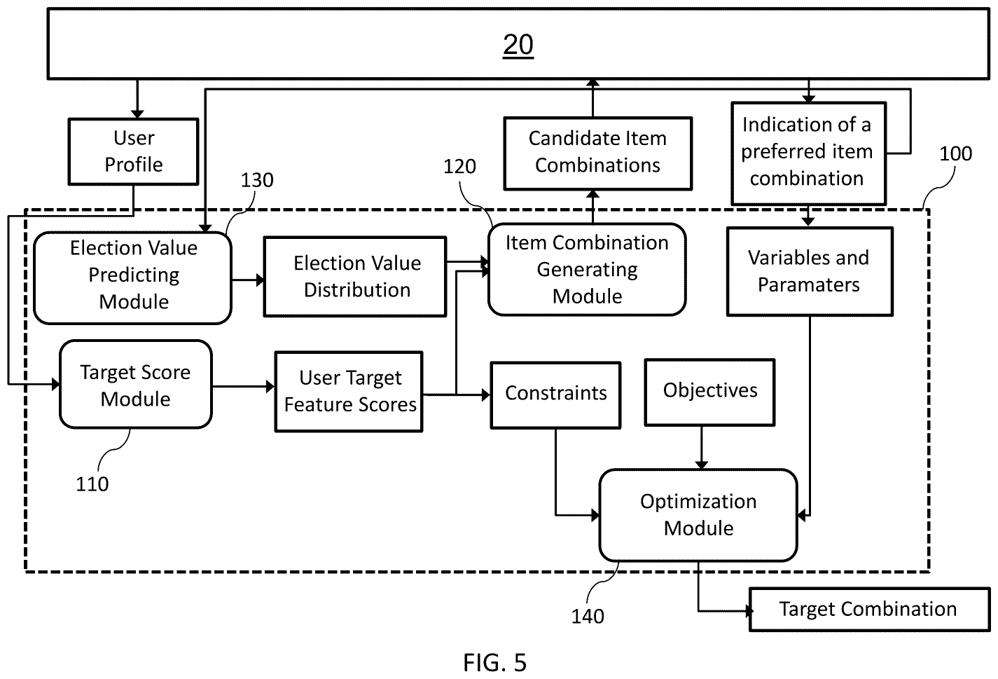

More specifically, FIG. 5 depicts modules of the recommendation system 100 and interactions with the user device 20 to determine the personalized target item combination in accordance with some non-limiting implementations of the present technology. The recommendation system 100 includes a target score module 110 that receives the user profile as an input. In use, the target score module 110 determines a user category for the user based on information included in the user profile. For example and without limitations, the target score module 110 may determine a matching score between the user profile and the user categories, the user category of the user being having the highest matching score. The target score module 110 further retrieves the target feature scores of the user category of the user, said target feature scores being further referred to as “user target feature scores”.

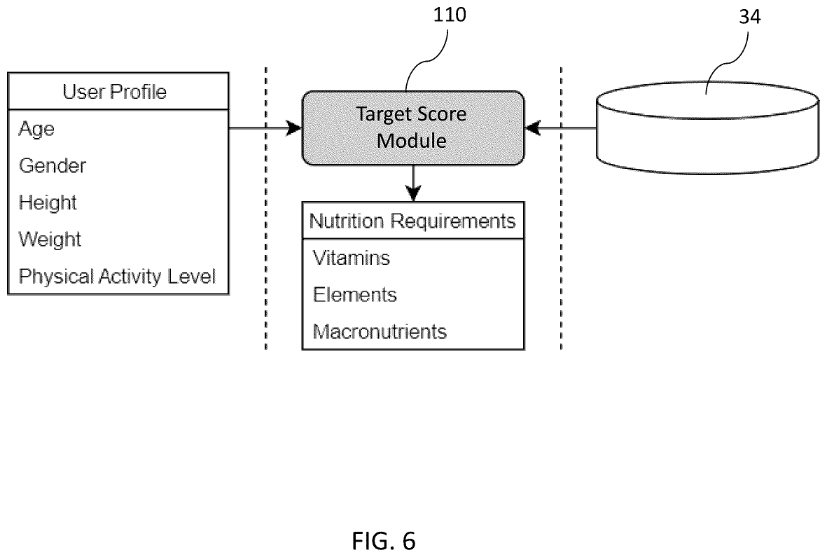

Operation of the target score module 110 is best shown on FIG. 6. As shown, the target score module 110 receives the user profile from the user device 20. Based on information included in the user profile, the target score module 110 identifies a user category of the user and fetches the target feature scores for the user form the identified user category from the target feature score database 34. The target feature scores of a user may be referred to as “user target feature scores”. In the illustrative example of FIG. 6, the user target feature scores are indicative of nutrition requirements as set by health organizations (e.g. the World Health Organization).

Referring back to FIG. 5, the user target feature scores are further transmitted to an item combination generating module 120 of the recommendation system 100. In use, the item combination generating module 120 generates a list of candidate item combinations. For example, each candidate item may include three items. Other numbers of item per item combination are contemplated in alternative implementations. In this implementation, each candidate item combination includes a meal tagged as a breakfast, a meal tagged as a lunch and a meal tagged as a dinner. Broadly speaking, each combination includes one item from each item category.

More specifically, the item combination generating module 120 takes the user target feature scores and an election value distribution associated with the user as inputs, to retrieve items from the item database 32 and generate the list of candidate item combinations.

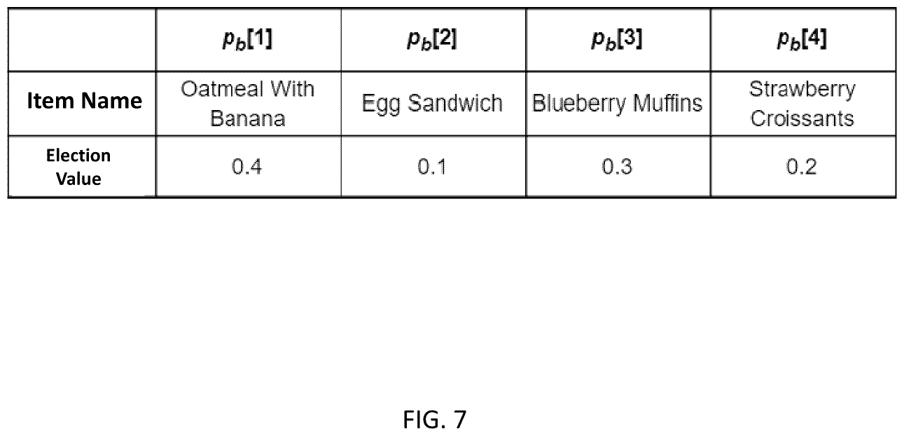

As shown on FIG. 5, the recommendation system 100 includes an election value predicting module 130 to determine the election values of the items. As described herein before, each item of the item database 32 may be associated with an election score indicative of a user preference of the user for that item. FIG. 7 shows a plurality of item of the item database and election values associated therewith. In the illustrative example of FIG. 7, it can be said that the user for which the election values are represented would prefer an oatmeal with banana over an egg sandwich, and blueberry muffins over strawberry croissants. The election value predicting module 130 may use a neural network to determine an election value for each item based on an indication of preference of the user for a set of items such as the items presented to the user during initialization.

The election value predicting module 130 may employ the neural network to determine election value distributions based on indication of preferences of the user for sub-items (e.g. ingredients of the meals) and/or for items of the item database 32.

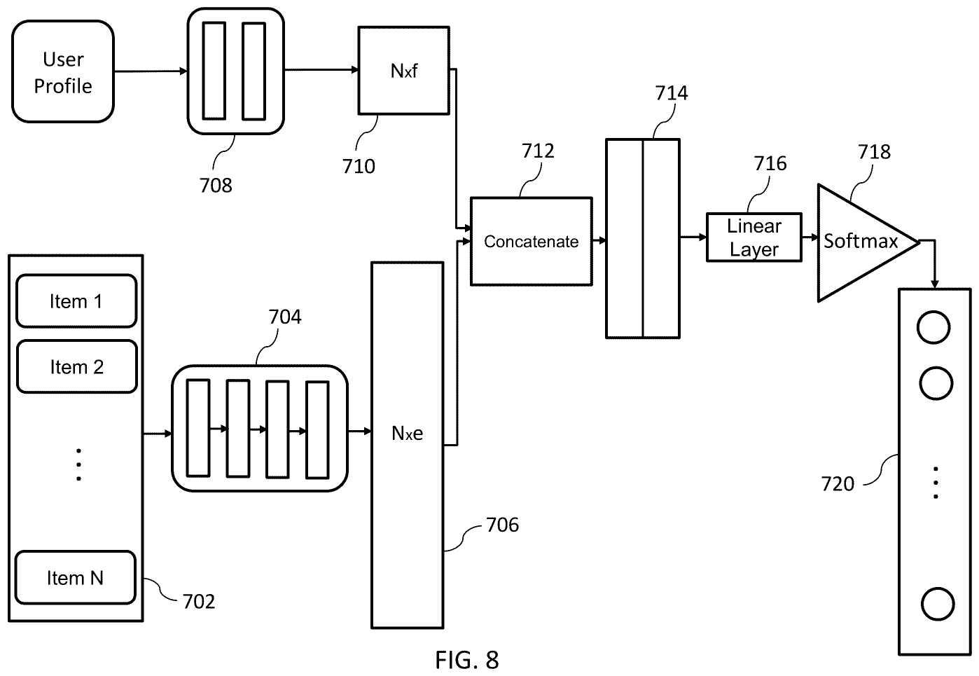

For example FIG. 8 shows a diagram of operations executed by a neural network of the election value predicting module 130 to determine election values according to some implementations of the present technology. In this implementation, a training phase of the neural network may include recording preferences of the user for a set of items. This may be performed by presenting a set of M items from the database 32 through the user device 20 and receiving indication of a preferred item among the M sampled items from the user.

For example, the item selected as the preferred item among the M sampled items may receive an election value of 1 and the other sampled items receive an election value of 0. It can be said that the neural network may use training datasets that include a training input including a set of items and a given user profile, and a training label including the preferred item selected by the user among the set of items. During a training iteration, the training label can be further compared to an output of the neural network such that errors of identification of the preferred item may be backpropagated to update the parameters of the neural network.

Broadly speaking, ground-truth about preferences of a user (i.e. the election values) may be determined upon receiving indication about the user's preferences when presented different items. In some implementations, ground-truth about preferences of a user may be determined based on preferences of another user with a similar user profile or a dissimilar user profile. For example, a first user having a first user profile may be assigned with election values identical to election values associated with a second user having a second user profile, the first and second user profiles being similar (e.g. an average of the differences between each field of information of the user profiles such as age, gender, weight, socio-economic status is below a pre-determined threshold). As another example, a third user having a third user profile may be assigned with election values being inverses of election values associated with a fourth user having a fourth user profile, the third and fourth user profiles being dissimilar (e.g. an average of the differences between each field of information of the user profiles such as age, gender, weight, socio-economic status is above a pre-determined threshold).

In at least some implementations of the present technology, the comparison of the output of the neural network during training against the training label may be performed by employing a loss function for determining a “loss” that is used for adjusting the neural network during the respective training iteration. Illustrative examples of loss functions include cross-entropy loss function or any other suitable loss function.

Preferred items for different users may be used to train the neural network. For example, the training datasets may include a training input including the set of items a plurality of user profiles. The election values of the items during training of the neural network may be averages of the election values assigned by the users.

In an implementation, an inference phase of the neural network includes receiving the user profile and a set 702 of N candidate items by the neural network. More specifically, the election value predicting module 130 may employ an item encoder 704 and a profile encoder 708 to map the set 702 and the user profile respectively. As such, the item encoder 704 provides a set 706 of Nxe item embeddings while the profile encoder 708 provides a Nxf user profile embedding 710.

In an implementation, the election value predicting module 130 employs a concatenating module 712- to concatenate the item embeddings and the user profile embedding into a concatenation 714. The concatenation 714 is further passed through a linear layer and a Softmax function to obtain a set 720 of item election values for the items of set 702.

In the same or other implementations, the election value predicting module 130 determines an election value distribution for each item category. In this example, an election value distribution p_{b} is determined for item tagged as breakfasts, an election value distribution l_{b} is determined for item tagged as lunches and an election value distribution p_{d} is determined for item tagged as dinners. Each election value distribution defines the election values of the corresponding items. In some implementations, the sum of all values in a meal preference distribution should be 1.

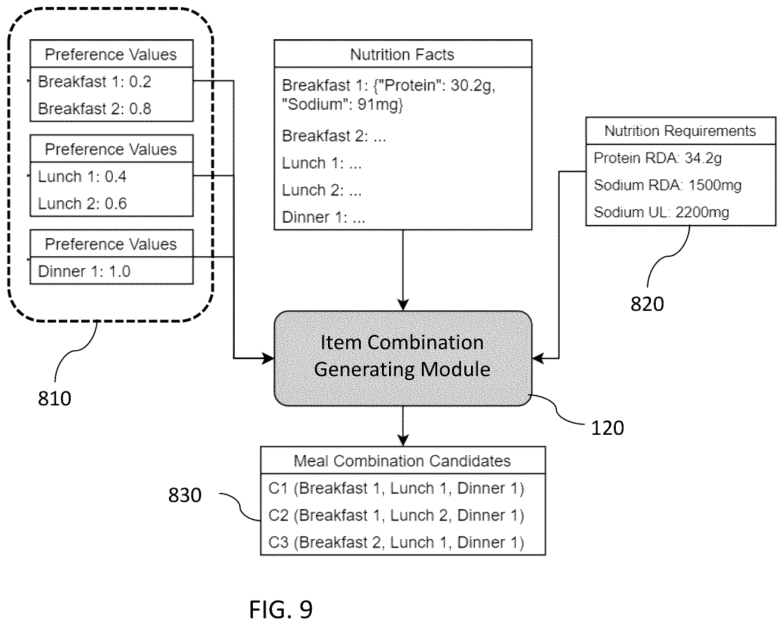

With respect to FIG. 9, the item combination generating module 120 takes user target feature scores 810 (i.e. the nutrition requirements for the user in the illustrative example of personalized optimization-based meal plan recommendation application), item election values distributions 820 (i.e. the meal preference distributions) and the items of the item database 32 as inputs. The item combination generating module 120 generates a list 830 of candidate item combinations therefrom.

In some implementations, the item combination generating module 120 employs a heuristic algorithm to establish the list 830 by maximizing the global election values of the candidate item combinations while meeting requirements for the global feature scores. For example and without limitations, the item combination generating module 120 may generate the list 830 by exhaustively examining all combinations (each combination including one item from each item category). For example, if there are 100 breakfast meals, 90 lunch meals and 80 dinner meals, there will be 100*90*80 combinations.

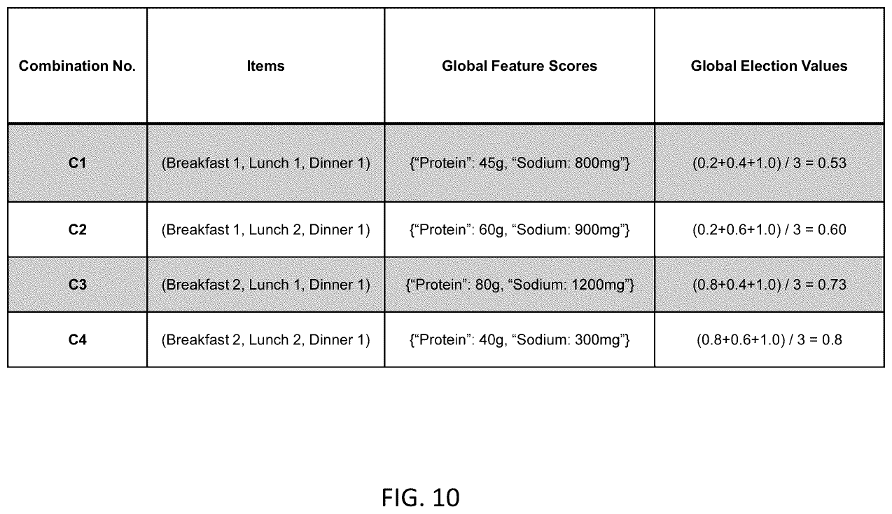

FIG. 10 is a representation of the list 830 of four candidate item combinations C1, C2, C3 and C4 generated by the item combination generating module 120. In some implementations, the feature scores of the of the items of a same item combination are added together to form a global feature score of the item combination. For example, the feature scores relative to the features “Protein” and “Sodium” are added for each item combination. The global features scores are further compared to corresponding user features scores determined by the target score module 110. In some implementations, the target feature scores may include a minimum threshold value and a maximum threshold value for a given feature. In response to determining that an item combination has a feature whose global feature score is out of a range defined between the minimum threshold value and the maximum threshold value for that feature, the candidate item combination may be removed from the list 830. In the item combination C4, the global feature score for the feature “Sodium” is 300 mg, which is below a minimum threshold value of 500 mg for example. The candidate item combination C4 may thus be removed from the list 830. In the illustrative example of application of personalized optimization-based meal plan recommendation, the minimum threshold value may be 50% of a recommended value of a corresponding nutrient (RDAs), and the maximum threshold value may be an optional upper bound value (ULs).

In some implementations, the election values of the item of a same item combination are averaged to determine a global election value of the item combination. For example, the item election value of a first item of the combination C1 is 0.2, the item election value of a second item of the combination C1 is 0.4 and the item election value of a third item of the combination C1 is 1. The global election value for the combination C1 is thus 0.53. The global election value may alternatively be a median value or a weighted average of the item election values or derived from the item election values in any suitable manner. In some implementations, the recommendation system 100 may remove candidate item combinations from the list 830 in response to their global election values being below a pre-determined threshold.

In the personalized optimization-based meal plan recommendation application, an item combination should satisfy the daily nutritional needs while maximizing the sum of meal preference values. It should be noted that a quantity of the sub-items (e.g. the ingredients) is still set to default values in the list 830 and will be updated as described in greater details hereinafter.

In some implementations, the item combination generating module 120 determines the candidate item combinations of the list 830 by maximizing the global election value thereof.

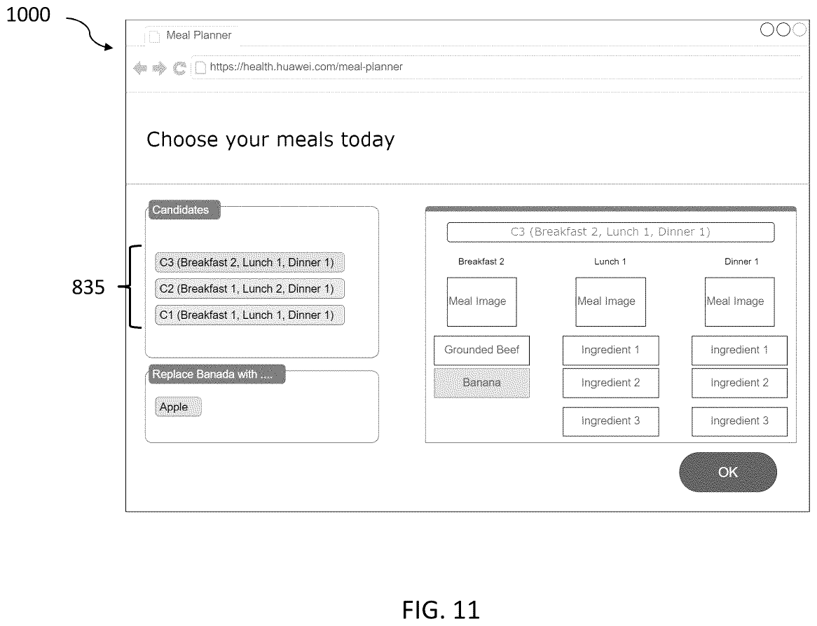

Referring back to FIG. 5, the list 830 or a portion thereof is further transmitted to the user device 20. In some implementations, the list 830 is order by global election value and a top portion (e.g. the first ten candidate item combinations) having thus the highest global election values is presented to the user. FIG. 11 is a representation of an interface 1000 of the user device to present a top portion 835 of the list 830 to the user. Information about the items of the candidate item combinations is rendered on the interface to provide details to the user about the candidate item combinations.

The recommendation system 100 further receives an indication of a preferred item combination among the candidate item combinations of the list 830. For example, the user may choose and select and candidate item combination using an interface of the user device. In some implementations, the user may add/replace/delete sub-items of the item of the candidate item combinations. The recommendation system 100 may replace a sub-item by another sub-item. Said other sub-item may have a same quantity unit in the item database 32 (e.g., grams, cup, tsp). After replacing the ingredient, the recommendation system 100 assesses whether or not the global feature scores of the adjusted preferred item combination fulfill the requirements of the target feature scores.

It should be noted that indication of the preferred item combination among the candidate item combinations of the list 830 is further used by the server to update the election value predicting module 130. More specifically, the election value distributions may be updated based on said indication to have a more accurate representation of the taste and preferences of the user.

Referring back to FIG. 5, the recommendation system 100 further includes an optimization module 140 for generating a personalized target item combination based on the preferred candidate item combination. Broadly speaking, the optimization module 140 updates a quantity of the sub-items of the items of the preferred candidate item combination to form the personalized target item combination.

In some implementations, the optimization module 140 receives a first input including the items of the preferred candidate item combination. The amount of the sub-items of said items are processed as variable and the associated feature scores of each sub-item are processed as parameters. In the illustrative example of personalized optimization-based meal plan recommendation, each nutrient have its own constraints (e.g., nutritional needs) and the optimization module 140 calculates appropriate ingredient amounts to make sure that each nutrient constraint is satisfied.

The optimization module 140 receives a second input including of constraints: 1) upper bound constraints; 2) percent constraints. The upper bound constraints can be directly constructed with the output (e.g., ULs) from the target score module 110. The percent constraints may be pre-determined values stored in the target score database. For example, the percent constraints may be set as defined in nutrition guidelines to balance energy contribution between ingredients of the meal.

The optimization module 140 receives a third input including the target feature scores as set in the target feature score database 34. The amount of sub-items may be denoted (X₁, X₂, . . . , Xₙ) and feature A (e.g., vitamin A) values in sub-items are (Y^{A}₁, Y^{A}₂, . . . , Y^{A}ₙ). In use, the optimization module 140 minimizes the objective function (1) defined as:

$${\sum }_{A ∈ Features} \frac{\vert {R}^{A} - {\sum }_{i = 1}^{n} {X}_{i} \times {Y}_{i}^{A} \vert}{{R}^{A}}$$

where R^{A} represents the target feature score for feature A. In the illustrative example of personalized optimization-based meal plan recommendation, minimizing the objective function may result in determining amount of ingredients that produce a meal plan that has minimum distance to the recommended nutrition requirements.

It can be said that the optimization module 140 converts nutrition needs and finalized meal combination to constraints, parameters and variables, and further uses a customizable objective function to determine the quantities of each ingredient in three meals and thus determines the personalized target item combination.

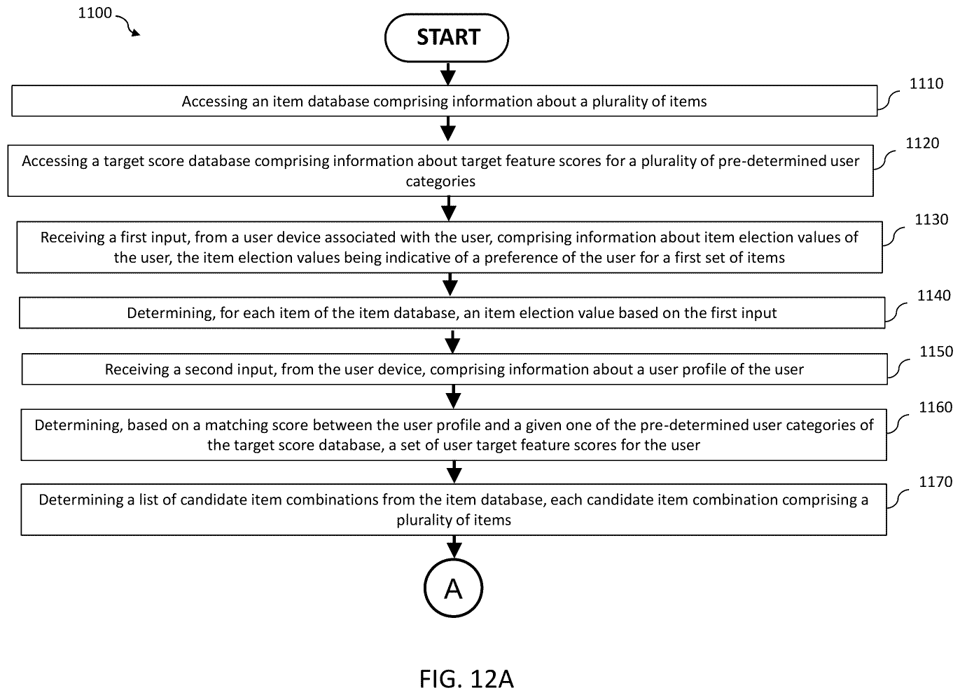

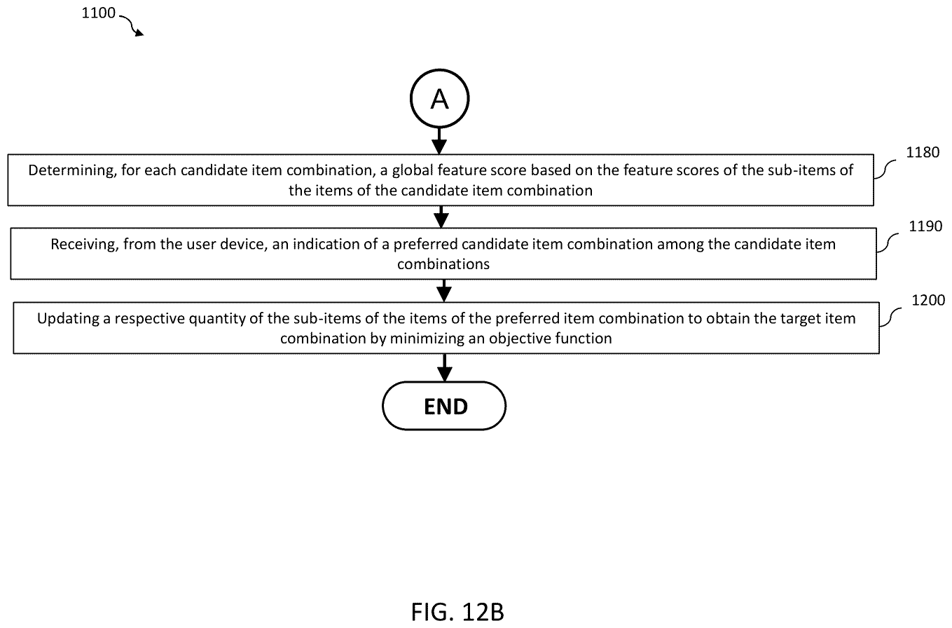

FIGS. 12A and 12B are flow diagrams of a computer-implemented method 1100 for generating a personalized target combination of items for a user according to some embodiments of the present technology. In one or more aspects, the method 1100 or one or more steps thereof may be performed by a processor or a computer system, such as the computing unit of the recommendation system 100. The method 1100 or one or more steps thereof may be embodied in computer-executable instructions that are stored in a computer-readable medium, such as a non-transitory mass storage device, loaded into memory and executed by a CPU. Some steps or portions of steps in the flow diagram may be omitted or changed in order.

The method 1100 includes accessing, by a server at operation 1110, an item database such as the item database 32. The item database includes information about a plurality of items, each item being associated with one or more sub-items composing the item, each sub-item being associated with one or more feature scores for one or more corresponding features of the sub-item. In some implementations, each item of the item database includes a tag indicative of an item category of the item, the item database comprising three categories of items. For example and without limitations, each item may map information about a meal, each sub-item may map information about an ingredient of the meal, each feature score may map information about a nutrition value of the corresponding ingredient, and each feature may map information about a nutrient of the corresponding ingredient.

The method 1100 includes accessing, by the server at operation 1120, a target score database such as the target feature score database 34. The item database includes comprising information about target feature scores for a plurality of pre-determined user categories.

The method 1100 includes receiving, by the server at operation 1130, a first input including information about item election values of the user. The first input is received from a user device that may be the user device 20. The item election values are indicative of a preference of the user for a first set of items.

The method 1100 includes determining, by the server at operation 1140, an item election value based on the first input for each item of the item database. In some implementations, the server may employ a neural network to determine, based on the item election values of the first set of items, an item election value for each of the other items of the item database to determine the item election values based on the first input. The neural network may be employed to determine a sub-item election value for each sub-item of the item database.

The method 1100 includes receiving, by the server at operation 1150, a second input including information about a user profile of the user from the user device.

The method 1100 includes determining, by the server at operation 1160, a set of user target feature scores for the user based on a matching score between the user profile and a given one of the pre-determined user categories of the target score database.

The method 1100 includes determining, by the server at operation 1170, a list of candidate item combinations from the item database, each candidate item combination comprising a plurality of items. For example, each candidate item combination may include three items. The items selected to form the list of candidate item combinations may be selected in a second set of items having a corresponding election score above a pre-determined threshold.

The method 1100 includes determining, by the server at operation 1180, a global feature score based on the feature scores of the sub-items of the items of the candidate item combination for each candidate item combination.

The method 1100 includes receiving, by the server at operation 1190, an indication of a preferred item combination among the candidate item combinations from the user device.

The method 1100 includes updating, by the server at operation 1200, a respective quantity of the sub-items of the items of the preferred item combination to obtain the personalized target item combination by minimizing the objective function (1), namely:

$${\sum }_{A ∈ Features} \frac{\vert {R}^{A} - {\sum }_{i = 1}^{n} {X}_{i} \times {Y}_{i}^{A} \vert}{{R}^{A}}$$

where Xᵢ is a quantity of the sub-item i, Yᵢ^{A} is a feature value of the sub-item i for the feature A and R^{A} is a target feature score for the feature A as defined in the target score database for the user.

In some implementations, the method 1100 further includes ordering the list of candidate item combinations based on global election value of the candidate item combinations. In the same or other implementations, the global election value of each candidate item combination is determined based on election values associated with items of the candidate item combination. The method 1100 may further include providing to the user device a top portion of the list of candidate item combinations (e.g. the first ten candidate item combinations).

The method 1100 may further includes updating the item election values of the items of the item database based on the indication of the preferred item combination among the candidate item combinations. The server may further transmit information about the personalized target item combination to the user.

While the above-described implementations have been described and shown with reference to particular steps performed in a particular order, it will be understood that these steps may be combined, sub-divided, or re-ordered without departing from the teachings of the present technology. At least some of the steps may be executed in parallel or in series. Accordingly, the order and grouping of the steps is not a limitation of the present technology.

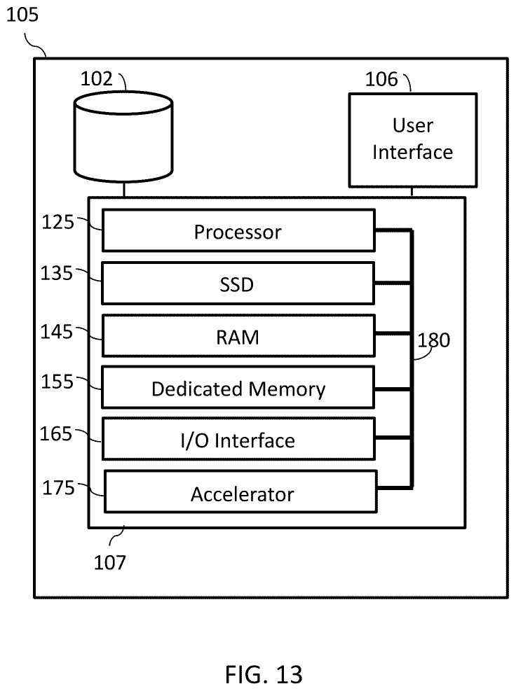

With reference to FIG. 13, there is shown an electronic device 105 in accordance with non-limiting implementations of the present technology. The recommendation system 100 and the user device 20 may implemented as the electronic device 105. The electronic device 105 includes a computing unit 107. In some embodiments, the computing unit 107 may be implemented by any of a conventional personal computer, a controller, and/or an electronic device (e.g., a server, a controller unit, a control device, a monitoring device etc.) and/or any combination thereof appropriate to the relevant task at hand. In some embodiments, the computing unit 107 includes various hardware components including one or more single or multi-core processors collectively represented by a processor 125, a solid-state drive 135, a RAM 145, a dedicated memory 155 and an input/output interface 165. The computing unit 107 may be a generic computer system.

In some other embodiments, the computing unit 107 may be an “off the shelf” generic computer system. In some embodiments, the computing unit 107 may also be distributed amongst multiple systems. The computing unit 107 may also be specifically dedicated to the implementation of the present technology. As a person in the art of the present technology may appreciate, multiple variations as to how the computing unit 107 is implemented may be envisioned without departing from the scope of the present technology.

Communication between the various components of the computing unit 107 may be enabled by one or more internal and/or external buses 185 (e.g. a PCI bus, universal serial bus, IEEE 1394 “Firewire” bus, SCSI bus, Serial-ATA bus, ARINC bus, etc.), to which the various hardware components are electronically coupled.

The input/output interface 165 may provide networking capabilities such as wired or wireless access. As an example, the input/output interface 165 may include a networking interface such as, but not limited to, one or more network ports, one or more network sockets, one or more network interface controllers and the like. Multiple examples of how the networking interface may be implemented will become apparent to the person skilled in the art of the present technology. For example, but without being limitative, the networking interface may implement specific physical layer and data link layer standard such as Ethernet, Fibre Channel, Wi-Fi or Token Ring. The specific physical layer and the data link layer may provide a base for a full network protocol stack, allowing communication among small groups of computers on the same local area network (LAN) and large-scale network communications through routable protocols, such as Internet Protocol (IP).

According to implementations of the present technology, the solid-state drive 135 stores program instructions suitable for being loaded into the RAM 145 and executed by the processor 125. Although illustrated as a solid-state drive 135, any type of memory may be used in place of the solid-state drive 135, such as a hard disk, optical disk, and/or removable storage media.

The processor 125 may be a general-purpose processor, such as a central processing unit (CPU) or a processor dedicated to a specific purpose, such as a digital signal processor (DSP). In some embodiments, the processor 125 may also rely on an accelerator 170 dedicated to certain given tasks. In some embodiments, the processor 125 or the accelerator 170 may be implemented as one or more field programmable gate arrays (FPGAs). Moreover, explicit use of the term “processor”, should not be construed to refer exclusively to hardware capable of executing software, and may implicitly include, without limitation, application specific integrated circuit (ASIC), read-only memory (ROM) for storing software, RAM, and non-volatile storage. Other hardware, conventional and/or custom, may also be included.

Further, the electronic device 105 may include a Human-Machine Interface (HMI) 106. The HMI 106 may include a screen or a display capable of rendering an interface such as the interface 1000 and/or any other information suitable for performing the methods described herein. In this embodiment, the display of the HMI 106 includes and/or be housed with a touchscreen to permit users to input data via some combination of virtual keyboards, icons, menus, or other Graphical User Interfaces (GUIs). The HMI 106 may thus be referred to as a user interface 106. In some embodiments, the display of the user interface 106 may be implemented using a Liquid Crystal Display (LCD) display or a Light Emitting Diode (LED) display, such as an Organic LED (OLED) display. The device may be, for example and without being limitative, a handheld computer, a personal digital assistant, a cellular phone, a network device, a smartphone, a navigation device, an e-mail device, a game console, or a combination of two or more of these data processing devices or other data processing devices. The user interface 106 may be embedded in the electronic device 105 as in the illustrated embodiment of FIG. 13 or located in an external physical location accessible to the user. For example, the user may communicate with the computing unit 107 (i.e. send instructions thereto and receive information therefrom) by using the user interface 106 wirelessly connected to the computing unit 107. The computing unit 107 may be communicate with the user interface 106 via a network (not shown) such as a Local Area Network (LAN) and/or a wireless connexion such as a Wireless Local Area Network (WLAN).

The electronic device 105 may include a memory 102 communicably connected to the computing unit 107 for storing target feature scores for example, and/or history of determined personalized target item combinations. The memory 102 may be embedded in the electronic device 105 as in the illustrated embodiment of FIG. 13 or located in an external physical location. The computing unit 107 may be configured to access a content of the memory 102 via a network (not shown) such as a Local Area Network (LAN) and/or a wireless connexion such as a Wireless Local Area Network (WLAN).

The electronic device 105 may also include a power system (not depicted) for powering the various components. The power system may include a power management system, one or more power sources (e.g., battery, alternating current (AC)), a recharging system, a power failure detection circuit, a power converter or inverter and any other components associated with the generation, management and distribution of power in mobile or non-mobile devices.

It should be noted that the computing unit 107 may be implemented as a conventional computer server or cloud-based (or on-demand) environment. Needless to say, the computing unit 107 may be implemented in any other suitable hardware, software, and/or firmware, or a combination thereof. In the depicted non-limiting embodiments of the present technology in FIG. 13, the computing unit 107 is a single server. In alternative non-limiting embodiments of the present technology, the functionality of the computing unit 107 may be distributed and may be implemented via multiple servers.

Those skilled in the art will appreciate that processor 125 is generally representative of a processing capability that may be provided by, for example, a Central Processing Unit (CPU). In some embodiments, in place of or in addition to one or more conventional CPUs, one or more specialized processing cores may be provided. For example, one or more Graphic Processing Units (GPUs), Tensor Processing Units (TPUs), accelerated processors (or processing accelerators) and/or any other suitable processing unit may be provided in addition to or in place of one or more CPUs. In this embodiment, the processor 125 of the computing unit 107 is a Graphical Processing Unit (GPU) and the dedicated memory 155 is a Video Random access Memory (VRAM) of the processing unit 120. In alternative embodiments, the dedicated memory 155 may be a Random Access Memory (RAM), a Video Random Access Memory (VRAM), a Window Random Access Memory (WRAM), a Multibank Dynamic Random Access Memory (MDRAM), a Double Data Rate (DDR) memory, a Graphics Double Data Rate (GDDR) memory, a High Bandwidth Memory (HBM), a Fast-Cycle Random-Access Memory (FCRAM) or any other suitable type of computer memory.

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

What is claimed is:

1. A computer-implemented method for generating a personalized target combination of items for a user, the computer-implemented method comprising:
accessing an item database comprising information about a plurality of items, each item being associated with one or more sub-items composing the item, each sub-item being associated with one or more feature scores for one or more corresponding features of the sub-item;
accessing a target score database comprising information about target feature scores for a plurality of pre-determined user categories;
receiving a first input, from a user device associated with the user, comprising information about item election values of the user, the item election values being indicative of a preference of the user for a first set of items;
determining, for each item of the item database, an item election value based on the first input;
receiving a second input, from the user device, comprising information about a user profile of the user;
determining, based on a matching score between the user profile and a given one of the pre-determined user categories of the target score database, a set of user target feature scores for the user;
determining a list of candidate item combinations from the item database, each candidate item combination comprising a plurality of items;
determining, for each candidate item combination, a global feature score based on the feature scores of the sub-items of the items of the candidate item combination;
receiving, from the user device, an indication of a preferred candidate item combination among the candidate item combinations; and
updating a respective quantity of the sub-items of the items of the preferred item combination to obtain the personalized target item combination by minimizing:
$${\sum }_{A ∈ Features} \frac{\vert {R}^{A} - {\sum }_{i = 1}^{n} {X}_{i} \times {Y}_{i}^{A} \vert}{{R}^{A}}$$
where Xᵢ is a quantity of the sub-item i, Yᵢ^{A} is a feature value of the sub-item i for the feature A and R^{A} is a target feature score for the feature A as defined in the target score database for the user.

2. The computer-implemented method of claim 1, further comprising:
ordering the list of candidate item combinations based on global election value of the candidate item combinations, the global election value of each candidate item combination being determined based on election values associated with items of the candidate item combination; and
providing to the user device a top portion of the list of candidate item combinations.

3. The computer-implemented method of claim 1, further comprising updating the item election values of the items of the item database based on the indication of the preferred item combination among the candidate item combinations.

4. The computer-implemented method of claim 1, wherein determining, for each item of the item database, an item election value based on the first input comprises:
employing a neural network to determine, based on the item election values of the first set of items, an item election value for each of the other item of the item database.

5. The computer-implemented method of claim 4, wherein the neural network is employed to determine a sub-item election value for each sub-item of the item database.

6. The computer-implemented method of claim 4, wherein determining the list of candidate item combinations from the item database comprises:
selecting a second set of items, the items of the second set having a corresponding election score above a pre-determined threshold.

7. The computer-implemented method of claim 1, wherein each candidate item combination comprises three items.

8. The computer-implemented method of claim 7, wherein each item of the item database comprises a tag indicative of an item category of the item, the item database comprising three categories of items.

9. The computer-implemented method of claim 1, wherein:
each item maps information about a meal,
each sub-item maps information about an ingredient of the meal,
each feature score maps information about a nutrition value of the corresponding ingredient, and
each feature maps information about a nutrient of the corresponding ingredient.

10. The computer-implemented method of claim 1, further comprising transmitting information about the personalized target item combination to the user.

11. A system for generating a personalized target combination of items for a user, the system comprising a controller and a memory storing a plurality of executable instructions which, when executed by the controller, cause the controller to:
access an item database comprising information about a plurality of items, each item being associated with one or more sub-items composing the item, each sub-item being associated with one or more feature scores for one or more corresponding features of the sub-item;
access a target score database comprising information about target feature scores for a plurality of pre-determined user profiles;
receive, from a user device associated with the user, a first input comprising information about item election values of the user, the item election values being indicative of a preference of the user for a first set of items;
determine, for each item of the item database, an item election value based on the first input;
receive, from the user device, a second input comprising information about a user profile of the user;
determine, based on a matching score between the user profile and a given one of the pre-determined user profiles of the target score database, a set of user target feature scores for the user;
determine a list of candidate item combinations from the item database, each candidate item combination comprising a plurality of items;
determine, for each candidate item combination, a global feature score based on the feature scores of the sub-items of the item of the candidate item combination;
receive, from the user device, an indication of a preferred candidate item combination among the candidate item combinations; and
update a respective quantity of the sub-items of the items of the preferred item combination to obtain the personalized target item combination by minimizing:
$${\sum }_{A ∈ Nutrients} \frac{\vert {R}^{A} - {\sum }_{i = 1}^{n} {X}_{i} \times {Y}_{i}^{A} \vert}{{R}^{A}}$$
where Xᵢ is a quantity of the sub-item i, Yᵢ^{A} is a feature value of the sub-item i for the feature A and R^{A} is a recommended feature score for the feature A as defined in the target score database for the user.

12. The system of claim 11, further configured to:
order the list of candidate item combinations based on global election value of the candidate item combinations, the global election value of each candidate item combination being determined based on election values associated with items of the candidate item combination; and
provide to the user device a top portion of the list of candidate item combinations.

13. The system of claim 11, further configured to adjust the item election values of the items of the item database based on the indication of the preferred item combination among the candidate item combinations.

14. The system of claim 11, wherein determining, for each item of the item database, an item election value based on the first input comprises:
employing, by the controller, a neural network to determine, based on the item election values of the first set of items, an item election value for each of the other item of the item database.

15. The system of claim 14, wherein the neural network is employed to determine a sub-item election value for each sub-item of the item database.

16. The system of claim 14, wherein determining the list of candidate item combinations from the item database comprises:
selecting, by the controller, a second set of items, the items of the second set having a corresponding election score above a pre-determined threshold.

17. The system of claim 11, wherein each item combination comprises three items.

18. The system of claim 17, wherein each item of the item database comprises a tag indicative of an item category of the item, the item database comprising three categories of item.

19. The system of claim 11, wherein:
each item maps information about a meal,
each sub-item maps information about an ingredient of the meal,
each feature score maps information about a nutrition value of the corresponding ingredient, and
each feature maps information about a nutrient of the corresponding ingredient.

20. The system of claim 11, further configured to transmit information about the personalized target item combination to the user.
